(19) United States
(12) Patent Application Publication
(10) Pub. No.: US 2026/0049997 A1
(43) Pub. Date: Feb. 19, 2026

(54) PROTEIN MARKER PANEL FOR SCREENING CLINICAL FUTILE RECANALIZATION (FR) IN LARGE VESSEL OCCLUSION-INDUCED ACUTE ISCHEMIC STROKE (LVO-AIS) AND USE THEREOF

(71) Applicant: NANJING FIRST HOSPITAL, Nanjing City (CN)

(72) Inventors: Qiwen DENG, Nanjing City (CN); Teng JIANG, Nanjing City (CN); Lin ZHU, Nanjing City (CN); Shuo LI, Nanjing City (CN); Huiling SUN, Nanjing City (CN); Junshan ZHOU, Nanjing City (CN); Zhenzhen LI, Nanjing City (CN); Lifan JI, Nanjing City, (CN); Yiting ZHANG, Nanjing City (CN)

(21) Appl. No.: 19/291,971

(22) Filed: Aug. 6, 2025

(30) Foreign Application Priority Data
Aug. 15, 2024 (CN) 202411118826.8

(51) Int. Cl. G01N 33/68

(52) U.S. Cl. CPC G01N 33/6893; G01N 2333/521; G01N 2800/2871; G01N 2800/52

(57) ABSTRACT

A protein marker panel for screening clinical futile recanalization (FR) in patients with large vessel occlusion-induced acute ischemic stroke (LVO-AIS) and use thereof are provided. The protein marker panel includes GUCA2A, CCL14, STAB1, and VSIG4. A combined diagnostic value of the protein marker panel is high, and receiver operating characteristic (ROC) curve analysis demonstrates an efficacy in distinguishing and screening FR patients, with an area under the curve (AUC) of 0.959, indicating desirable sensitivity and specificity. Such a protein marker panel shows promise as a significant indicator for clinical decision-making in FR evaluation.

### CROSS REFERENCE TO RELATED APPLICATION

This patent application claims the benefit and priority of Chinese Patent Application No. 202411118826.8 filed with the China National Intellectual Property Administration on Aug. 15, 2024, the disclosure of which is incorporated by reference herein in its entirety as part of the present application.

### TECHNICAL FIELD

The present disclosure belongs to the technical field of biomedicine and molecular biology, and specifically relates to a protein marker panel for screening clinical futile recanalization (FR) in large vessel occlusion-induced acute ischemic stroke (LVO-AIS) and use thereof.

### BACKGROUND

Acute ischemic stroke (AIS) accounts for approximately 85% of all strokes and is the second leading cause of preventable death globally, which can result in permanent disability or death, posing a significant public health challenge worldwide. Approximately 46% of the AIS cases are caused by acute intracranial large vessel occlusion (LVO). Reperfusion therapies including intravenous thrombolysis and endovascular treatment (EVT) are critical for AIS management, in which EVT significantly reduces mortality in LVO-induced cerebral infarction and improves AIS prognosis. However, despite timely and successful recanalization of the occluded vessels, nearly half of LVO-AIS patients undergoing EVT still exhibit poor functional recovery. This phenomenon is termed “futile recanalization (FR)”. Multiple meta-analyses indicate that advanced age, high National Institutes of Health Stroke Scale (NIHSS) scores, delayed EVT, multiple mechanical thrombectomy attempts, pre-operative comorbidities (e.g., hypertension, diabetes), elevated blood glucose, collateral compensation status, IL-6 level, and other factors correlate with FR risk. These factors may predict FR occurrence, guide EVT decision-making, and thus reducing FR incidence through optimizing patient's selection for treatment, shortening recanalization time, minimizing endovascular attempts, and improving post-operative management of blood pressure and glucose. Nevertheless, the complex interplay of these factors is clinically challenging to control, which generally leads to rapid deterioration of the patients' condition or even death, severely impeding early intervention and functional rehabilitation in stroke patients. Consequently, FR imposes substantial economic burdens on families and society, emerging as an urgent clinical problem in cerebrovascular disease. Precise pre-operative FR prediction is thus essential for informing individualized treatment strategies. Yet, accurate early predictive markers for FR remain lacking. Identifying specific early-warning diagnostic factors for FR could enable individualized precision therapy in EVT planning.

To date, the pathophysiological mechanisms of FR remain unclear. Several studies report independent associations between FR and biomarkers such as neutrophil-to-lymphocyte ratio, platelet-to-lymphocyte ratio, matrix metalloproteinase-9, tenascin-C, thioredoxin, ADAMTS13, and gelsolin. A cohort study has found that in patients receiving only intravenous thrombolysis, those achieving recanalization within 2 h post-treatment exhibit higher baseline ADAMTS13 activity than non-recanalized patients. For those undergoing EVT, ADAMTS13 levels less than 982 ng/mL serve as an independent predictor of FR. However, the relationship between blood biomarkers (as practical tools) and FR pathophysiology remains insufficiently studied.

Proteins act as executors of life activities; alterations in their abundance and function may directly reflect an organism's physiological or pathological state. Proteomics employing high-throughput technologies may investigate the composition, function, and interactions of all proteins within a specific type of cells or tissues. This approach may offer biological elucidations for FR pathogenesis. Therefore, establishing an early screening model of biomarkers on the basis of proteomics, identifying and detecting peripheral blood protein biomarkers in FR patients at baseline, may facilitate early FR screening and provide individualized therapeutic decisions.

A proteomic study directs to jugular vein blood from anterior circulation LVO-AIS patients before and after successful recanalization after EVT has identified 13 differentially expressed proteins pre-EVT and 23 differentially expressed proteins post-EVT between effective recanalization (ER) and FR groups. These proteins participate in diverse processes, including: protein degradation/folding, lipogenesis or atherosclerosis, inflammatory responses, blood-brain barrier integrity maintenance, angiogenesis and intercellular adhesion, autophagy, neuroendocrine secretion, electrolyte homeostasis, vesicular trafficking, glucose metabolism, redox reactions, and cell growth/proliferation, migration, or apoptosis. Proteomics may deepen understanding of FR mechanisms and provide molecular markers for early FR prediction, offering a theoretical basis for optimizing treatment strategies. However, dedicated FR screening indicators are currently absent. Thus, discovering novel molecular markers holds significant importance for early FR screening, timely warning, and enhancing individualized treatment strategies.

In summary, while intravenous thrombolysis and EVT are pivotal for vascular recanalization, EVT is feasible for LVO-AIS patients coincident the tissue time window, even may reduce disability in large core infarction cases, however, EVT incurs high costs. Approximately 50% of patients experience FR, yet no definitive biological or neuroimaging biomarkers exist to predict the occurrence of FR. This imposes a substantial economic burden on both patients and society.

### SUMMARY

To address the deficiencies in the prior art, the present disclosure provides a protein marker panel for screening clinical futile recanalization (FR) in patients with large vessel occlusion-induced acute ischemic stroke (LVO-AIS) and use thereof. Biological markers capable of predicting FR are screened using high-throughput proteomics. These markers can effectively predict the occurrence of FR, provide an individualized strategy for selecting treatment options in LVO-AIS patients, and significantly reduce the economic burden on patients.

The present disclosure is implemented by the following technical solutions:

The present disclosure provides a protein marker panel for screening clinical FR in LVO-AIS patients, including GUCA2A, CCL14, STAB1, and VSIG4.

The present disclosure further provides use of the protein marker panel in preparation of a product for screening clinical FR in patients with LVO-AIS.

In some embodiments, the product includes a reagent or a kit for specifically detecting an expression level of the protein marker panel in a biological sample.

In some embodiments, the biological sample is peripheral blood from a subject.

In some embodiments, the reagent includes antibodies capturing human GUCA2A, CCL14, STAB1, and VSIG proteins.

The present disclosure further provides a kit for screening clinical FR in LVO-AIS patients, including a detection reagent, where the detection reagent includes antibodies capturing human GUCA2A, CCL14, STAB1, and VSIG proteins.

In some embodiments, the kit is an enzyme-linked immunosorbent assay (ELISA) detection kit.

The present disclosure further provides use of the protein marker panel in preparation of a product for assessing prognosis in an LVO-AIS patient.

In some embodiments, the product includes a reagent or a kit for detecting an expression level of the protein marker panel by ELISA.

The present disclosure has the following beneficial effects:
(1) The detection sample required by the present disclosure is patient peripheral blood. Peripheral blood samples are readily accessible. This minimizes time and effort, is critical in acute cerebral infarction patients for whom “time is brain”, and provides a robust theoretical basis for clinical decision-making.
(2) The present disclosure develops a novel protein marker panel for FR screening and diagnosis, which offers a new reference for FR screening.
(3) A combined diagnostic value of the protein marker panel is high. Receiver operating characteristic (ROC) curve analysis demonstrates an efficacy in distinguishing and screening FR patients, with an area under the curve (AUC) of 0.959, indicating desirable sensitivity and specificity. Such a protein marker panel shows promise as a significant indicator for clinical decision-making in FR evaluation.

### BRIEF DESCRIPTION OF THE DRAWINGS

FIGS. 1A-1D show proteomic analysis the expression of GUCA2A (FIG. 1A), CCL14 (FIG. 1B), STAB1 (FIG. 1C), and VSIG4 (FIG. 1D) between the ER and FR groups (N_{ER}=5, N_{FR}=5);

FIGS. 2A-2D show large-sample validation of GUCA2A (FIG. 2A), CCL14 (FIG. 2B), STAB1 (FIG. 2C), and VSIG4 (FIG. 2D) expression between the ER and FR groups (N_{ER}=80, N_{FR}=80); and

FIG. 3 shows ROC curve analysis of the combined diagnostic performance by GUCA2A, CCL14, STAB1, and VSIG4.

### DETAILED DESCRIPTION OF THE EMBODIMENTS

The present disclosure will be further described below in conjunction with the accompanying drawings and specific examples.

Unless otherwise specified, the technical means used in the following examples are conventional means well known to those skilled in the art. Experimental methods without specific conditions are all routine methods in the art.

### Example 1

In the initial phase of this example, FR screening was conducted in patients with LVO-AIS who underwent EVT to select 5 FR patients and 5 ER patients; their plasma samples were selected for the analysis on the changes in the plasma protein profiling of peripheral blood samples using 4D-DIA quantitative proteomics technology to screen for differentially expressed proteins. Ultimately, four protein molecules were selected: guanylate cyclase activator 2A (GUCA2A), chemokine (C—C motif) ligand 14 (CCL14), stabilin-1 (STAB1), and V-set and immunoglobulin domain containing 4 (VSIG4). This selection was then validated in an expanded sample size. Combined with statistical analysis, these markers were used for FR screening. The specific methodological steps were as follows:
1. Study Subjects and Samples

A total of 160 LVO-AIS patients who underwent successful recanalization after EVT at Nanjing First Hospital (Nanjing First Hospital Affiliated to Nanjing Medical University, Nanjing, Jiangsu province, China) between January 2021 and June 2023 were selected. All patients were diagnosed by 2 neurologists independently through clinical scale assessments and imaging examinations. At baseline, 3-5 mL of peripheral blood was collected from each of the patients into EDTA anticoagulant tubes. Plasma was separated via centrifugation within 30 min post-collection and stored at −80° C.
2. Patients were divided into ER and FR groups based on NIHSS and mRS scores (FR criteria: NIHSS score reduction <4 and mRS score >2 after 24 hours) 24-h post-recanalization. Among them, there were 80 ER patients and 80 FR patients.
3. Subsequent experiments were conducted according to the ELISA kit protocol. The operational procedures were as follows:
(1) Strips as required were taken out from the aluminum foil bag after 60-min equilibration at room temperature, and the remaining strips were put back in a ziplock bag, sealed and stored at 4° C.
(2) Standard, blank, and sample wells were designated. Into the standard wells 50 μL of serially diluted standards at different concentration were added.
(3) Into the sample wells 50 μL of test samples were added. 50 μL of sample dilution buffer was added into each of the blank wells.
(4) 100 μL of horseradish peroxidase (HRP)-conjugated detection antibody was added to all (blank/standard/sample) wells. Then the wells were sealed with adhesive film and incubated at 37° C. for 60 min to allow reaction.
(5) Liquid in the wells was discarded, and wells were blotted dry on absorbent paper. Each well was filled with 350 μL washing buffer, incubated for 1 min, then the washing buffer was discarded, and blotted again. This washing cycle was repeated for 5 times.
(6) 50 μL each of Substrate A and Substrate B were added to each well. Wells were incubated at 37° C. for 15 min in the dark.
(7) 50 μL of stop solution was added to each well. Optical density (OD) at 450 nm was measured within 15 min.
(8) A standard curve was plotted using OD values of the blank/standard wells. Protein concentration in the samples was calculated based on this curve. All samples in experimental and control groups were analyzed in triplicate.
4. Statistical Methods

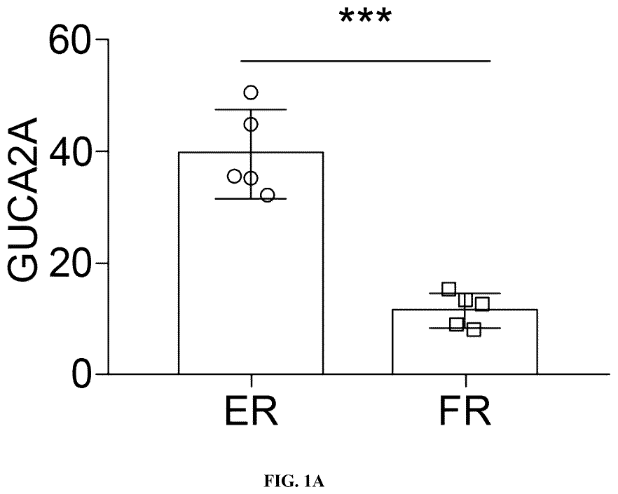

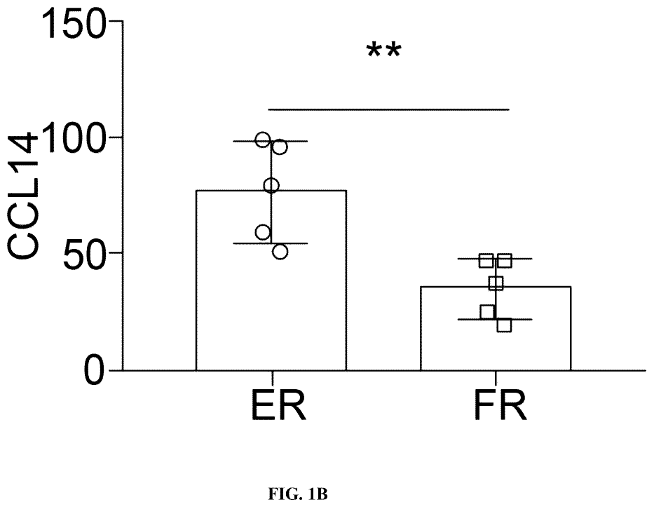

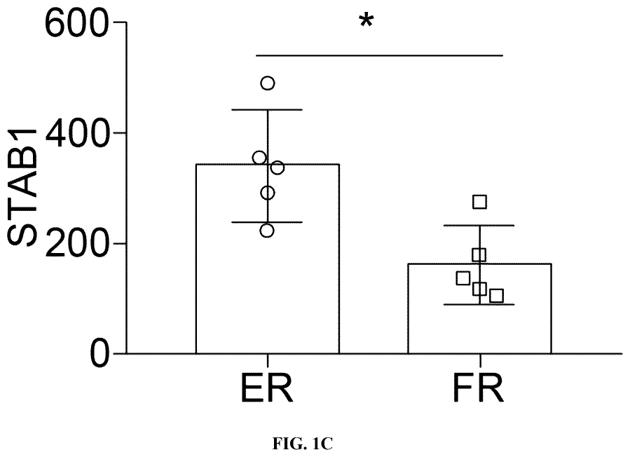

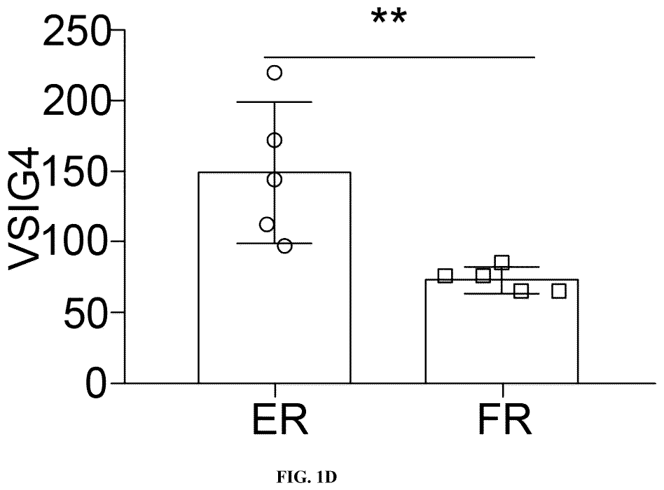

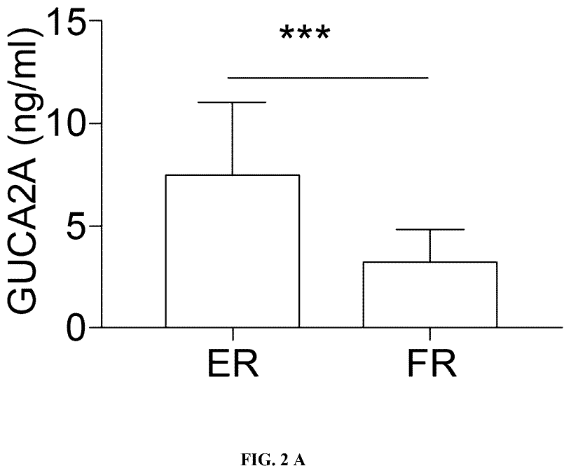

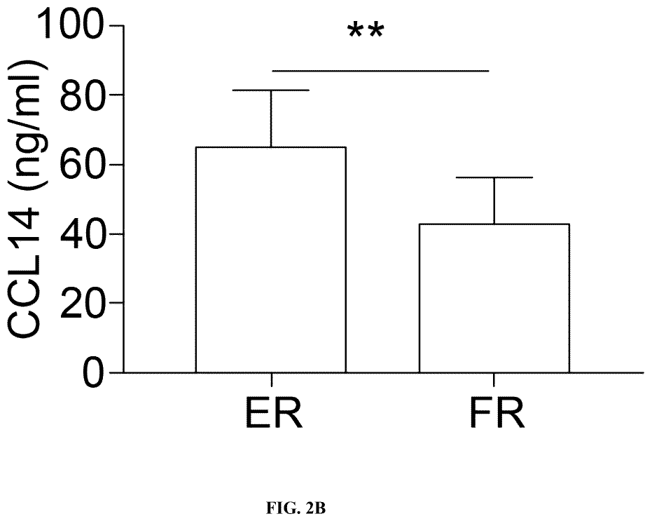

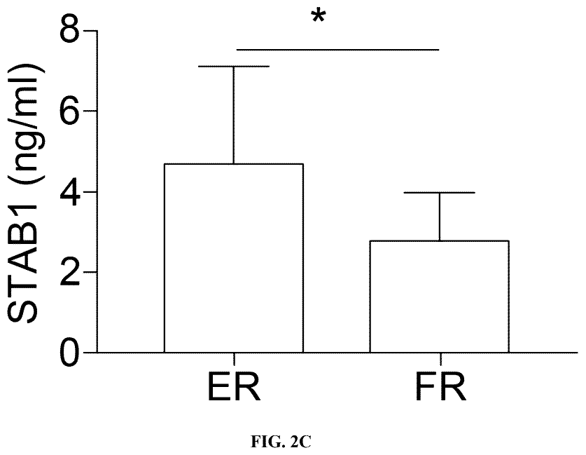

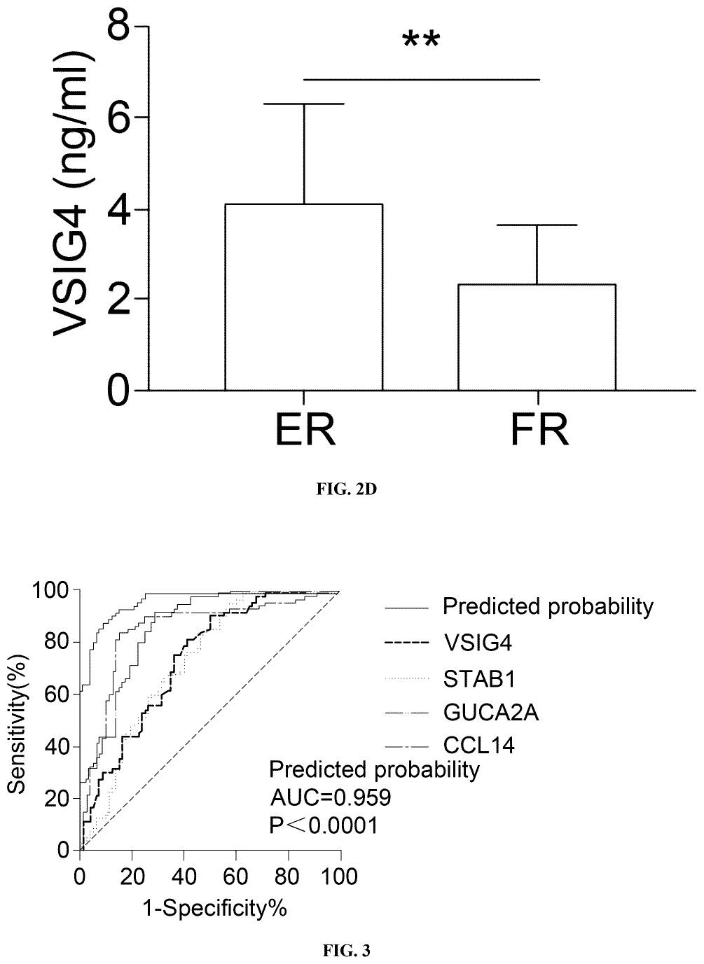

Data analysis was conducted using SPSS 25.0 software. Intergroup differences were assessed by t-test or rank sum test. ROC analysis was applied to evaluate the diagnostic value of individual biomarkers (GUCA2A, CCL14, STAB1, and VSIG4) and their combined diagnostic value.
5. Experimental Results
(1) Proteomic analysis as shown in FIGS. 1A-1D revealed significantly downregulated expression of GUCA2A, CCL14, STAB1, and VSIG4 in the FR group versus the ER group at baseline, where P_{GUCA2A}=0.00005, P_{CCL14}=0.005, P_{STAB1}=0.011, and P_{VSIG4}=0.009.
(2) ELISA validation the expression of GUCA2A, CCL14, STAB1, and VSIG4 in 80 each of ER and FR samples were shown in FIGS. 2A-2D, which confirmed consistent downregulation expression of GUCA2A, CCL14, STAB1, and VSIG4 in the FR group versus the ER group, where P_{GUCA2A}<0.0001, P_{CCL14}=0.004, P_{STAB1}=0.025, and P_{VSIG4}=0.006.

These above results described validated significantly reduced expression of all four biomarkers GUCA2A, CCL14, STAB1, and VSIG4 in FR subjects at baseline.
(3) The combined diagnostic performance of the differentially expressed proteins GUCA2A, CCL14, STAB1, and VSIG4 was measured by ROC analysis together with the calculation of the area under the ROC. The AUC, i.e., area under the ROC, is an index to characterize the diagnostic performance or accuracy. The closer AUC is to 1, the better the diagnostic performance is.

The results were shown in FIG. 3, where the AUC of the Predicted probability by the panel of GUCA2A, CCL14, STAB1, and VSIG4 is 0.959 (95% CI: 0.923-0.994, P<0.0001), demonstrating utility of GUCA2A, CCL14, STAB1, and VSIG4 as biomarkers for FR diagnosis.

The described examples are merely some rather than all of the examples of the present disclosure. The detailed description examples of the present disclosure are not intended to limit the protection scope of the present disclosure, but merely represent selected examples of the present disclosure. The protection scope of the present disclosure shall be subject to the scope required by the claims. All other examples obtained by those of ordinary skill in the art based on the examples of the present disclosure without creative efforts shall fall within the protection scope of the present disclosure.

What is claimed is:

1. A protein marker panel for screening clinical futile recanalization (FR) in large vessel occlusion-induced acute ischemic stroke (LVO-AIS), comprising GUCA2A, CCL14, STAB1, and VSIG4.

2. Use of a reagent for detecting the protein marker panel according to claim 1 in preparation of a product for screening clinical FR in LVO-AIS.

3. The use according to claim 2, wherein the product comprises a reagent or a kit for specifically detecting an expression level of the protein marker panel in a biological sample.

4. The use according to claim 3, wherein the biological sample is peripheral blood from a subject.

5. The use according to claim 3, wherein the reagent comprises human GUCA2A, CCL14, STAB1, and VSIG protein capture antibodies.

6. A kit for screening clinical FR in LVO-AIS, comprising a detection reagent, wherein the detection reagent comprises human GUCA2A, CCL14, STAB1, and VSIG protein capture antibodies.

7. The kit according to claim 6, wherein the kit is an enzyme-linked immunosorbent assay (ELISA) detection kit.
